(19) United States
(12) Patent Application Publication
(10) Pub. No.: US 2015/0018387 A1
(43) Pub. Date: Jan. 15, 2015

(54) HIGH CONCENTRATION LOCAL ANESTHETIC FORMULATIONS

(71) Applicant: Centrexion I, Inc., Baltimore, MD (US)

(72) Inventors: James N. Campbell, Baltimore, MD (US); Arthur F. Michaelis, Devon, PA (US)

(21) Appl. No.: 14/483,839

(22) Filed: Sep. 11, 2014

Related U.S. Application Data
(63) Continuation of application No. 12/192,871, filed on Aug. 15, 2008, now abandoned.
(60) Provisional application No. 60/956,458, filed on Aug. 17, 2007.

(51) Int. Cl. A61K 31/47; A61K 31/245; A61K 31/167; A61K 31/445

(52) U.S. Cl. CPC A61K 31/47; A61K 31/445; A61K 31/245; A61K 31/167
USPC 514/312; 514/330; 514/536; 514/626

(57) ABSTRACT

A transdermal topical anesthetic formulation, which can be used to ameliorate or inhibit neuropathic pain, has been developed. In the preferred embodiment, the topical anesthetic is a local anesthetic such as lidocaine, most preferably lidocaine free-base in a gel, and the dosage of the local anesthetic is effective in the painful area or immediately adjacent areas, to ameliorate or eliminate the pain. High concentration of local anesthetic in solution in the carrier is used to drive rapid release and uptake of the drug. Relief is typically obtained for a period of several hours.

### CROSS-REFERENCE TO RELATED APPLICATIONS

This application is a Continuation of U.S. Ser. No. 12/192,871, filed Aug. 15, 2008, which claims benefit of U.S. Ser. No. 60/956,458, entitled “Local Anesthetic Formulation for Treating Neuropathic Pain” by James N. Campbell, filed in the U.S. Patent and Trademark Office on Aug. 17, 2007. The entire contents of these applications are incorporated hereby by reference.

### TECHNICAL FIELD

The present invention relates to formulations containing a high concentration of topical anesthetic, such as lidocaine, which can be used for the treatment of neuropathic pain.

### BACKGROUND OF THE INVENTION

Neuropathic pain refers to pain that originates from pathology of the nervous system. Diabetes, infection (herpes zoster), nerve compression, nerve trauma, “channelopathies,” and autoimmune disease are examples of diseases that may cause neuropathic pain (Campbell and Meyer, Neuron, 52(1):77-92 (2006); Campbell, Muscle Nerve, 24:1261-1273 (2001)). Neuropathic pain is frequently chronic and may be the source of profound disability. Neuropathic pain is often associated with hyperalgesia (lowered pain threshold and enhanced pain perception) and/or by allodynia (pain from innocuous mechanical or thermal stimuli). Because of the often devastating consequences of neuropathic pain, and the limited efficacy of existing therapies, there has been intense interest in developing new therapies.

Classic analgesics have limited utility because of lack of efficacy or a high incidence of side effects. Data from clinical studies and conventional clinical wisdom indicate that NSAIDs are poorly effective. Opioids may be effective but side effects, tolerance, concern about addiction and diversion all limit their utility. A review analyzing the controlled clinical data for peripheral neuropathic pain (PNP) (Kingery, Pain, 73(2): 123-39 (1997) reported that NSAIDs were probably ineffective as analgesics for PNP and that there was no long-term data supporting the analgesic effectiveness of any drug. The results of published trials and clinical experience provide the foundation for specific recommendations for first-line treatments, which include gabapentin, 5% lidocaine patch, opioid analgesics, tramadol hydrochloride, and tricyclic antidepressants (reviewed by Vadalouca, et al., Ann NY Acad Sci., 1088:164-86 (2006).

Neuropathic pain has been shown to be sensitive to systemic delivery of anesthetics (Chabal, Anesthiology, (4):513-7 (1992). Neuropathic pain however is related at least in part to neural signals arising at the level of the skin (Sato and Perl, Science, 251(5001): 1608-10 (1991); Campbell and Meyer, Neuron, 52(1):77-92 (2006): Campbell, Muscle Nerve, 24:1261-1273 (2001)). Thus a clinical and scientific rationale exists for directing therapy directly to the skin.

Delivery of drugs by the transdermal route has been known for many years. Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir, matrix or monolithic devices are typically characterized by a falling drug flux with time, as the matrix layers closer to the skin are depleted of drug. Methods for making transdermal patches are described in U.S. Pat. Nos. 6,461,644, 6,676,961, 5,985,311, 5,948,433. With respect to lidocaine, U.S. Pat. Nos. 4,777,046, 5,958,446, 5,719,197, 5,686,099, 5,656,286, 5,474,783, 5,300,291, 4,994,267, 4,814,168, 7,018,647, 6,299,902; and 6,297,290 disclose compositions containing lidocaine in the range of 10-40% w/w which can be applied as a topical formulation (such as a patch).

Topical gels, plasters, and patches are described in U.S. Pat. Nos. 5,411,738, 5,601,838, 5,709,869 and 5,827,829 which are assigned to Endo Pharmaceuticals. The gels described in these patents contain from 2-20% lidocaine, preferably from 1-10% or 5-10% lidocaine.

A 5% lidocaine patch marketed as LIDODERM® is available from Endo Pharmaceuticals, Inc. The LIDODERM® patch comprises an adhesive material containing 5% lidocaine, which is applied to a non-woven polyester felt backing and covered with a polyethylene terephthalate (PET) film release liner. This patch is applied only once for up to 12 hours in a given 24 hour period. The marketed patch provides satisfactory therapy to some patients. Delivery of lidocaine in a patch, however, has numerous liabilities for the patient. Since the patch is a finite size and shape, the application area is determined by the patch and not by the dimensions of the painful site. If the area of pain is other than a large smooth surface, the patch may not necessarily fit the area or be comfortable to the wearer since the patch may not conform to the defect. For example, the patch is difficult to apply to toes and fingers. Applying the patch to the face creates a stigma issue for patients. The patch is undesirable for hair bearing areas as well since hair limits adhesiveness and because of the depillitation that may occur with removal of the patch. The patch may also make the patient warmer, and thus be a burden in hot environments.

The delivery of drug from the lidocaine patch is designed to be constant over the 12-hour exposure period. However, it may be therapeutically important to provide a loading dose of drug to eliminate pain quickly when first administering the therapy. It is well known in the treatment of pain that more analgesic is required to treat established pain than is needed to prevent pain from becoming more intense. Such a profile cannot be provided by a patch delivering at a constant rate.

It is therefore an object of the present invention to provide topical anesthetic formulations that can be used to provide relief from neuropathic pain over a period of time.

### SUMMARY OF THE INVENTION

A topical anesthetic formulation containing a high concentration of local anesthetic in a pharmaceutically acceptable carrier for topical application and method of use to ameliorate or inhibit pain, including neuropathic pain, has been developed, such that the target tissue (skin) is appropriately dosed with anesthetic. In the preferred embodiment, the local anesthetic is lidocaine, most preferably lidocaine free base, most preferably in a continuous phase gel, although creams, lotions, foams, sprays or ointments may also be used, and the dosage of the local anesthetic is effective in the painful area or immediately adjacent areas, to ameliorate or eliminate the pain. The formulations release the largest dose of drug shortly after administration, for example, from 0 to 6 hours after administration. This early time period release should result in a more rapid onset of pain relief for the patient. The concentration of the drug in the formulation is from about greater than 20% to about 40% or higher by weight of the formulation. In the preferred embodiment, the concentration is about 40%. The formulation is applied to the site of, or adjacent to, the painful area. Relief is typically obtained for a period of several hours or days, depending on the dosing schedule. The formulations can be applied once a day or more frequently, such as two times or three times a day. In a preferred embodiment, the formulation is applied for the treatment or alleviation of neuropathic pain.

### BRIEF DESCRIPTION OF THE DRAWINGS

FIG. 1 is a graph of the cumulative receptor phase levels of lidocaine from: gels ID:2749-30 (-♦-), ID:2749-32 (-▴-), ID:2749-31(-▪-), and ID:2749-28 (lidocaine HCl, -X-); spray ID:2749-72 (-Δ-); creams ID:2749-38 (-•-) and ID:2749-37 (-*-); foam ID:2749-52 (-∘-); compounding pharmacy product (-□-); and a lidocaine patch (-⋄-) through human skin, measured as micrograms lidocaine/cm², over time in hours.

### DETAILED DESCRIPTION OF THE INVENTION

### I. Formulations

The formulations contain high concentrations of drug applied in a continuous phase directly to the surface of affected skin. “High concentration”, as used herein, means that release of the drug is governed by the third law of thermodynamics; rather than Fick's Second Law of Diffusion, which governs the release of drug from dilute solutions. Fick's Second Law of diffusion instructs that the rate of release drug from dilute solutions. The result is that a large dose of drug is released in the early time period following administration, for example, 0-6 hours following administration. “High concentration” will typically be a concentration of greater than 20% drug/carrier w/w, as discussed in more detail below.

The formulation may be a single-phase system such as a gel or a more complex multiphasic system wherein one or more additional phases may be in dynamic equilibrium with the continuous phase. Examples of such systems include creams, lotions, emulsions of lipid containing droplets throughout a continuous aqueous phase, stable micellar dispersions, combinations of an emulsion with excess drug particles distributed throughout, and self-emulsifying systems. The common attribute of the various formulations would be the very high concentration of the drug in the continuous phase of the system.

As discussed above, Fick's Second Law of Diffusion governs release of drug from dilute solutions. However, Fick's law breaks down in very highly concentrated solution. In very highly concentrated solutions, the presence of the solute (i.e., drug) inhibits the ability of the solvent molecules to orient at will. Solvation of the drug in these highly concentrated solutions causes specific orientation of adjacent water molecules to a very high degree, creating a very high-energy state. Since the Third Law of Thermodynamics instructs that molecules will always seek a state of increased entropy in order to lower the overall energy of the system, there is an enhanced thermodynamic driving force to force the drug out of the continuous phase and across the barrier membranes of the skin. Removal of the drug from the continuous phase results in an increase in the entropy of the continuous phase as lowering concentration of the drug allows for more movement of the solvent molecules (i.e., increase in entropy) and, thus, an overall decrease in the energy of the system. The result is rapid early time delivery of the drug from the drug product to the target tissues.

Evidence of this effect can be seen in the data in the examples. The highly concentrated gel formulations provide higher early time (first six hours) levels of drug transport across the human skin membranes than does the reference lidocaine patch (5% drug content) or the lidocaine hydrochloride creams which have only very low effective levels of lidocaine free base (the uncharged base can cross the barrier membranes whereas the charged salt form can not).

A. Local Anesthetics

As used herein, the term “local anesthetic” means a drug which provides local numbness or pain relief. Local anesthetics cause reversible blockage of conduction and/or initiation of action potentials typically by actions related to the interference with voltage gated sodium channels. Lipid solubility appears to be the primary determinant of intrinsic anesthetic potency. Chemical compounds which are highly lipophilic tend to penetrate the nerve membrane more easily, such that fewer molecules are required for conduction blockade resulting in enhanced potency.

Chemically most local anesthetics are esters or amides. Esters include, but are not limited to, procaine, tetracaine, and chloroprocaine. They are hydrolyzed in plasma by pseudo-cholinesterase. Amides include, but are not limited to, lidocaine, mepivicaine, prilocaine, bupivacaine, and etidocaine. These compounds are often referred to as the “caine alkaloids”. Caine alkaloids generally have high first pass metabolisms. The liver rapidly metabolizes the drug and the kidneys excrete the metabolites and/or unchanged drug.

A number of different local anesthetics can be used, including dibucaine, bupivacaine, etidocaine, tetracaine, lidocaine, and xylocaine. In the preferred embodiment, the anesthetic is lidocaine, most preferably in the form of the free base, although it may be possible to use a salt, for example, the hydrochloride, hydrobromide, acetate, citrate, or sulfate salt. As demonstrated in the examples, gels containing lidocaine free base and creams and sprays containing lidocaine HCl were prepared. Compared to the free base form of these drugs, the more hydrophilic hydrochloride salt displays longer and denser nerve block, more complete release from matrices, slower clearance from the targeted nerve area, and less encapsulation.

The formulations described herein should deliver a high local concentration with little systemic absorption, which should minimize the adverse side effects associated with the systemic absorption of caine alkaloids. For example, alter administration of formulations containing lidocaine free base, little or no unchanged drug was detected in the plasma.

The formulations contain from about greater than 20% to about 60% of the drug by weight of the formulation. In the preferred formulation, the formulation contains about 40% by weight of lidocaine, most preferably of the free base. More of the salt form is required to achieve the same transdermal uptake, based on the studies in the following examples. The concentration and pharmacokinetics are dependent on the form of the local anesthetic and the excipient, as discussed in more detail below and demonstrated by the examples. In general, a lower concentration of lidocaine free base in a gel will provide equivalent uptake as a higher concentration of lidocaine HCl in a multiphasic excipient.

B. Excipients

1. Lotions, Creams, Gels, Ointments, Foams

“Water Soluble” as used herein refers to substances that have a solubility of greater than or equal to 5 g/100 ml water.

“Lipid Soluble” as used herein refers to substances that have a solubility of greater than or equal to 5 g/100 ml in a hydrophobic liquid such as castor oil.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible wit the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophyllic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes. Self generating emulsions are known to enhance the absorption of drugs as shown in the following table.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%. Examples of the composition of lidocaine/lidocaine hydrochloride creams are shown in the examples.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Examples of the composition of lidocaine/lidocaine hydrochloride gels are shown in the examples. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether, alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

### II. Methods of Treatment

A. Effective Dosages; Sites of Administration

The formulations described herein can be administered at or adjacent to the sites of pain to provide relief. The formulations can be administered once a day, for example, for fast, temporary pain relief, or more frequently, such as twice or three times a day, to maintain pain relief over an extended period of time.

1. Topical

The composition is applied topically to a site at or adjacent to a painful region. The composition is reapplied as necessary. The dosing is applied to the painful skin and subcutaneous structures in order to effect pain relief while avoiding the side effects associated with systemic delivery. Pain relief is obtained within minutes to hours and lasts for periods of approximately three to six hours to 24 hours. The compounds are applied such that the dosage is sufficient to provide an effective dose in the painful area or immediately adjacent areas, to ameliorate or eliminate pain and other unpleasant sensations such as itching.

2. Intradermal

Some of the formulation can be administered intradermally, using, for example, an insulin syringe. Care should be taken to administer the smallest dose possible, and in all cases, topical or intradermal, care should be taken to avoid systemic levels or local toxicity.

B. Therapeutic Indications

The formulations can be used to treat pain such as neuropathic pain. Neuropathic pain is pain that is associated with diseases or conditions that affect the nervous system primarily. For example, pain from osteoarthritis of the knee is not considered neuropathic pain. However, pain associated with diabetic neuropathy or pain associated with nerve injury is considered neuropathic. Neuropathic pain may also arise from disorders of ion channels, such as the sodium channels. The nervous system can generate and perpetuate pain (i.e., neuropathic), without any ongoing stimuli from injury. Neuropathic pain is often puzzling and frustrating for both patients and physicians because it seems to have no cause, responds poorly to standard pain therapies, can last indefinitely and even escalate over time, and often results in severe disability.

Four pathological mechanisms are associated with the generation of pain in peripheral tissues in neuropathic pain conditions. These are: 1) nociceptor sensitization, whereby nociceptors have enhanced sensitivity to stimuli; 2) spontaneous activity related either to abnormal activity of transduction channels, or abnormal sensitivity of spike generation mechanisms; 3) abnormal coupling between sympathetic efferent fibers and nociceptors (sympathetically maintained pain); and 4) deafferentation, a central mechanism of pain whereby pain results from abnormal activity in neurons concerned with pain in the central nervous system as a result of altered input from primary afferents.

The primary sensory neurons that carry signals related to pain are called C-fiber and A-delta nociceptors. Normally, they fire action potentials in response to noxious mechanical, thermal, and/or chemical stimuli. Lumbar disk herniation with its accompanying chemical irritants to the adjacent nerve root can produce sciatic nerve pain. Carpal tunnel syndrome is due to a combination of repetitive stretching of the median nerve, compression caused by edema and hypertrophy of surrounding tissues, and inflammation producing chemical irritation of the median nerve. Trigeminal neuralgia has been attributed to vascular compression on the trigeminal nerve near the brain stem and may also relate to conditions such as multiple sclerosis.

Nerve fibers that have been damaged by injury or disease can fire spontaneously at the site of injury or at ectopic foci along the damaged nerve. Resulting paroxysms of pain are often described as lancinating, stabbing, or shooting. It is believed that when many nerve fibers are affected and fire asynchronously, neuropathic pain has a quality of continuous burning results. In addition however, the nerve fibers that share the innervation territory of the injured nerve can also discharge abnormally. This discharge arises in the skin and therefore lends itself to topical therapy. Clonidine applied topically has been discovered to relieve pain after delivery to the painful site, for example.

Under normal conditions, sensations are transmitted from peripheral tissues via a connected chain of neurons in the spinal cord, brain stem, and brain. Interruption of any portion of that chain provides the potential for increased irritability and firing of nerves further up the pathway. This phenomenon explains how phantom limb pain can occur: Loss of sensory input from a limb can produce spontaneous firing of second- and third-order neurons, resulting in pain and other sensory experiences in the missing limb. Similarly, nerves damaged by diabetic neuropathy, post-herpetic neuropathy, or peripheral nerve trauma may generate firing in the higher-order nerves and, thus, ongoing pain.

### EXAMPLES

The present invention will be further understood by reference to the following non-limiting examples.

### Example 1

### Determination of Solubility and Compatibility of Lidocaine and Lidocaine HCl in Pharmaceutically Acceptable Topical Carrier

The primary goal was to develop a fast-acting topical product containing 40% Lidocaine as the Active Pharmaceutical Ingredient (API) with limited systemic exposure for the treatment of neuropathic pain.

Materials and Methods

The solubility and compatibility of lidocaine and lidocaine HCl in solvents typically used in topical pharmaceutical products was assessed in order to direct the formulation development efforts. The solvents were selected based on anticipated solubility parameters and solvent behavior, and their inclusion on the FDA approved Inactive Ingredient Guide (IIG). Additional attributes included the ability to accommodate a high level of drug while retaining adequate cosmetic properties, and the potential for a quick-drying product for application to the torso and face.

Initially, the solubility of lidocaine and lidocaine hydrochloride was evaluated in single solvents with varying lipophilicity. Given the high concentration of API, the solubilized drug samples were visually inspected following a week of storage to ensure no crystallization had occurred. Based on the single solvent data, a compatibility study was initiated to evaluate the chemical stability of the drug at a concentration of 40% w/w in a variety of solvent blends that would form the base of potential prototype gel or cream formulations. Both lidocaine and lidocaine hydrochloride retained their physical appearance and the absence of a drop in potency between the two week samples stored at accelerated conditions versus the initial samples supported the chemical compatibility of lidocaine in the solvent blends.

TABLE 1
R&D Stability Summary

| Batch # | | | | Assay | | |
|---|---|---|---|---|---|---|
| (Type) | T₀ | Conditions | pH | (API % LC) | Viscosity/cP | Comment |
| 2749-28 | pH = 5.54 | Freeze/Thaw | 5.78 | 104.4 | ND | |
| (Lidocaine | Assay = 99.6 | Hot/Cold | 5.69 | 105.6 | ND | |
| HCL GEL) | Viscosity = ND | 1 month @ 5° C. | 5.82 | 103.3 | ND | |
| | | 1 month @ 25° C. | 5.80 | 104.8 | ND | |
| | | 1 month @ 40° C. | 5.77 | 104.6 | ND | |
| | | 3 months @ 5° C. | 5.54 | 102.4 | ND | |
| | | 3 months @ 25° C. | 5.62 | 102.9 | 5400* | |
| | | 3 months @ 40° C. | 5.71 | 102.9 | ND | |
| 2749-30 | pH = 9.35 (1:9) | Freeze/Thaw | 8.75 (1:9) | 102.7 | ND | |
| (Lidocaine | Assay = 104.2 | Hot/Cold | 8.97 (1:9) | 105.8 | ND | |
| GEL) | Viscosity = ND | 1 month @ 5° C. | 8.91 (1:9) | 104.5 | ND | |
| | | 1 month @ 25° C. | 8.79 (1:9) | 102.6 | ND | |
| | | 1 month @ 40° C. | 8.81 (1:9) | 102.9 | ND | |
| | | 3 months @ 5° C. | ND | 105.1 | ND | |
| | | 3 months @ 25° C. | 9.43 (1:9) | 105.4 | 3750* | |
| | | 3 months @ 40° C. | ND | 102.4 | ND | |
| 2749-31 | pH = 9.40 (1:9) | Freeze/Thaw | 9.13 (1:9) | 101.8 | ND | |
| (Lidocaine | Assay = 99.2 | Hot/Cold | 9.15 (1:9) | 104.3 | ND | |
| GEL) | Viscosity = ND | 1 month @ 5° C. | 9.20 (1:9) | 100.5 | ND | |
| | | 1 month @ 25° C. | 9.22 (1:9) | 102.8 | ND | |
| | | 1 month @ 40° C. | 9.26 (1:9) | 101.0 | ND | |
| | | 3 months @ 5° C. | ND | 103.4 | ND | |
| | | 3 months @ 25° C. | 9.70 (1:9) | 99.7 | 4375* | |
| | | 3 months @ 40° C. | ND | 100.6 | ND | |
| 2749-32 | pH = 9.40 (1:9) | Freeze/Thaw | 9.20 (1:9) | 104.5 | ND | |
| (Lidocaine | Assay = 99.8 | Hot/Cold | 9.30 (1:9) | 101.3 | ND | |
| GEL) | Viscosity = ND | 1 month @ 5° C. | 9.36 (1:9) | 101.3 | ND | |
| | | 1 month @ 25° C. | 9.26 (1:9) | 101.0 | ND | |
| | | 1 month @ 40° C. | 9.28 (1:9) | 100.8 | ND | |
| | | 3 months @ 5° C. | ND | 103.4 | ND | |
| | | 3 months @ 25° C. | 9.80 (1:9) | 102.5 | 3650* | |
| | | 3 months @ 40° C. | ND | 101.7 | ND | |
| 2749-37 | pH = 5.55 | Freeze/Thaw | 5.87 | 104.6 | 3,095** | |
| (Lidocaine | Assay = 101.2 | Hot/Cold | 6.02 | 102.8 | 3,079** | |
| HCL | Viscosity = | 1 month @ 5° C. | 6.06 | 105.1 | ND | |
| Cream) | 2,658** (60 rpm) | 1 month @ 25° C. | 6.06 | 104.2 | ND | |
| | | 1 month @ 40° C. | 6.08 | 104.7 | ND | |
| | | 3 months @ 5° C. | ND | 99.6 | ND | Phase separation |
| | | 3 months @ 25° C. | 5.98 | 104.6 | 3300* | |
| | | 3 months @ 40° C. | 5.90 | 104.0 | ND | |
| 2749-38 | pH = 5.60 | Freeze/Thaw | 6.02 | 100.2 | ND | |
| (Lidocaine | Assay = 99.1 | Hot/Cold | 6.01 | 102.5 | ND | |
| HCL | Viscosity = ND | 1 month @ 5° C. | 6.04 | 108.3 | ND | |
| Cream) | | 1 month @ 25° C. | 6.07 | 101.8 | ND | |
| | | 1 month @ 40° C. | 6.08 | 101.4 | ND | |
| | | 3 months @ 5° C. | ND | 100.7 | ND | Phase separation |
| | | 3 months @ 25° C. | 5.92 | 101.0 | 2225* | |
| | | 3 months @ 40° C. | 5.90 | 103.0 | ND | |
| 2749-42 | pH = 5.54 | Freeze/Thaw | 5.13 | 99.2 | ND | |
| (Lidocaine | Assay = ND | Hot/Cold | 5.20 | 99.7 | ND | |
| HCL | Viscosity = ND | 1 month @ 5° C. | 5.17 | 99.6 | ND | |
| Spray) | | 1 month @ 25° C. | 5.23 | 100.6 | ND | |
| | | 2.5 months @ 40° C. | 5.26 | 99.8 | ND | |
| | | 3 months @ 5° C. | 5.52 | 103.2 | ND | |
| | | 2.5 months @ 25° C. | 5.50 | 100.6 | ND | |
| | | 2.5 months @ 40° C. | 5.41 | 103.3 | ND | |
| 2749-52 | pH = 5.63 | Freeze/Thaw | 5.20 | 102.8 | ND | |
| (Lidocaine | Assay = ND | Hot/Cold | 5.22 | 101.0 | ND | |
| HCL | Viscosity = ND | 1 month @ 5° C. | 5.21 | 101.4 | ND | |
| Foam) | | 1 month @ 25° C. | 5.22 | 101.7 | ND | |
| | | 1 month @ 40° C. | 5.20 | 102.0 | ND | |
| | | 2.5 months @ 5° C. | 5.42 | 104.3 | ND | |
| | | 2.5 months @ 25° C. | 5.50 | 101.3 | ND | |
| | | 2.5 months @ 40° C. | 5.53 | 102.7 | ND | |

*Measured with Brookfield viscometer, Spindle used: 27, Sample weight: 12.5 gm, rpm = 50
**Measured with Brookfield viscometer, Spindle used: 14, Sample weight: 2.5 gm, rpm = 60

TABLE 2A
Composition of creams with Lidocaine HCl as the active agent;
Compositions of creams with lidocaine as the active agent

| | | Wt % | | | |
|---|---|---|---|---|---|
| | IIG | Batch | Batch | Batch | |
| Ingredients | Max. | #9 | #10 | #11 | Function |
| Lidocaine HCL | N/A | 40 | 40 | 40 | Active |
| Propylene glycol | 98 | 5.0 | 5.0 | 5.0 | Solvent/Delivery Agent |
| Glycerin | 50 | — | — | 3.0 | Humectant |
| Water | N/A | qs(43.5) | qs(43.5) | qs(38.1) | Solvent |
| Hydroxyethylcellulose250- | 4.0 | 1.0 | 1.0 | 1.0 | Gelling Agent |
| HHA-Pharm | | | | | |
| Diisopropyl adipate | 20 | — | 10 | — | Oil Phase |
| Oleyl alcohol | 10 | — | — | 5.0 | Oil Phase |
| Light Mineral oil | 95 | 10 | — | 7.0 | Oil Phase |
| Pemulen TR1 (Carbomer | 0.3 | 0.3 | 0.3 | 0.3 | Emulsifier |
| 1342) | | | | | |
| Span 80 (Sorbitan | 7.0 | — | — | 0.4 | Emulsifier |
| monooleate) | | | | | |
| Methylparaben | 70.0 | 0.17 | 0.17 | 0.17 | Preservative |
| Propylparaben | 30.0 | 0.03 | 0.03 | 0.03 | Preservative |
| | IIG | Wt % | | | |
| Ingredients | Max. | Batch #5 | Batch #6 | Function | |
| Lidocaine | N/A | 40 | 40 | Active | |
| Isopropyl myristate | 35 | 10.0 | 10 | Solvent/Emollient | |
| Diisopropyl adipate | 20 | 10 | — | Solvent/Delivery | |
| | | | | Agent | |
| Myristyl lactate | 92 | qs(18.1) | qs(21.1) | Solvent/Delivery | |
| | | | | Agent | |
| Oleic acid | 7.4 | — | 7 | Solvent/Delivery | |
| | | | | Agent | |
| Hydroxypropyl cellulose, | 4.0 | 1.0 | 1.0 | Gelling Agent | |
| HSV Pharm | | | | | |
| Pemulen TR2 (Carbomer | 0.3 | 0.3 | 0.3 | Emulsifier | |
| 1342) | | | | | |
| Polysorbate 80 (Tween | 9.4 | 0.4 | 0.4 | Emulsifier | |
| 80) | | | | | |
| Methylparaben | 70.0 | 0.17 | 0.17 | Preservative | |
| Propylparaben | 30.0 | 0.03 | 0.03 | Preservative | |
| Propylene glycol | 98 | 1.0 | 1.0 | Solvent | |
| Water | N/A | 19 | 19 | Vehicle | |

TABLE 2B
Composition of gels with Lidocaine HCl as the active agent;
Composition of gels with Lidocaine as the active agent

| | |
|---|---|
| IIG | Wt % |

| Ingredients | Max. | Batch #7 | Batch #8 | Function |
|---|---|---|---|---|
| Lidocaine HCL | NA | 40 | 40 | Active |
| Benzyl alcohol | 50 | 2 | 2 | Preservative |
| Propylene glycol | 98 | 5 | 10 | Solvent/Delivery |
| | | | | Agent |
| Glycerin | 50 | 3 | — | Humectant |
| Sorbitol, 70% | 67.52 | — | 3 | Humectant |
| Isopropyl alcohol | 99 | — | 10 | Solvent |
| Water | N/A | qs (49) | qs (34) | Solvent |
| Hydroxyethyl | 1.25 | 1.0 | 1.0 | Gelling Agent |
| cellulose, | | | | |
| 250-HHX-Pharm | | | | |

| | | Wt % | | | | |
|---|---|---|---|---|---|---|
| | II | Batch | Batch | Batch | Batch | |
| Ingredients | Max. | #1 | #2 | #3 | #4 | Function |
| Lidocaine | N/A | 40 | 40 | 40 | 40 | Active |
| Isopropyl myristate | 35 | — | — | — | 10 | Solvent/Emollient |
| Transcutol P | 25 | 10 | 10 | 20 | 10 | Solvent/Emollient |
| Propylene glycol | 98 | 20 | 10 | 10 | 20 | Solvent/Emollient |
| Dimethyl isosorbide | 15 | — | 10 | 10 | — | Solvent/Emollient |
| Ethyl acetate | 31 | — | 10 | — | — | Solvent/Emollient |
| Isopropyl alcohol | 99 | qs (18.8) | qs (18.8) | — | — | Solvent |
| Ethanol | 96 | — | — | qs (18.8) | qs (18.8) | Solvent |
| Water (purified) | N/A | 10.0 | | | | Vehicle |

A total of eleven formulations were prepared in which lidocaine or lidocaine hydrochloride was formulated at 40% w/w, shown in Tables 2A and 2B. Out of the 11 formulations, four contained the lidocaine free base (both non-aqueous and aqueous gels), while the remaining seven formulations contained the HCl salt form of lidocaine (cream, gel, spray, and foam dosage forms). The formulations were packaged in clear glass vials and stored at 5° C., 25° C., and 40° C. for a month. Separately, they were also subjected to three cycles each of freeze/thaw or hot/cold temperature cycling.

All prototype formulations were tested for potency of the active, appearance, and pH. Select samples were also submitted for viscosity and microscopy testing.

Results

Stability results are shown in Table 1.

The data show no significant loss of the active with time or temperature for all prototype formulations tested. No evidence of degradation products was observed.

None of the prototypes showed a change in appearance at any of the conditions tested with the exception of 2749-37 and 2749-38 which exhibited phase-separation when stored at 5° C. Prototype formulations containing the lidocaine base showed an increase in color (yellowish) intensity with an increase in storage temperature. Formulation 2749-30 had a strong odor, which can be attributed to the ethyl acetate it contains.

After storage for three months at 25° C., no precipitation or crystallization was observed. The pH values for the prototype formulations, with the exception of the gels listed below, ranged from 5.41 to 5.98. Target pH was 5.5 to 6.0. The measured pH represents no significant change from the initial value for all samples evaluated. Prototype gels containing lidocaine (2749-30 and 2749-32) are non-aqueous: therefore pH was measured following a 1:9 dilution with water in order to monitor changes to the pH of the diluted composition. The data show no significant change in measured pH value with time or temperature.

Select samples were evaluated for viscosity. None showed noticeable thinning and all samples maintained their gel-like or creamy consistency. Prototype 2749-28 exhibited some stringiness, which could be optimized by varying the concentration of the thickening agent in the product.

In summary, after one month of storage, the formulations were physically and chemically evaluated. All prototype formulations with the exception of the creams containing lidocaine HCl retained their initial physical and chemical stability properties after being stored at different conditions for one month. Based on the results generated at the 3-month time point, all prototypes with the exception of 2749-37 and 2749-38, qualify for further consideration. Prototype formulations 2749-37 and 2749-38 would require further optimization to address the phase separation observed at refrigerated conditions.

### Example 2

### In Vitro Percutaneous Absorption of Lidocaine from Prototype Formulations Using Human Skin

Materials and Methods

Based on the results of Example 1, eight prototype formulations were then selected and submitted for evaluation in an in vitro Skin Penetration Study.

The purpose of this study was to characterize the in vitro percutaneous absorption of the actives (lidocaine free-base or lidocaine HCl) from prototype formulations, compared to two control formulations (a compounding pharmacy product and a marketed patch, LIDODERM®), following topical application to excised human skin from elective surgery. Selection of the formulas to test in this study was based primarily on physical and chemical stability, a desire to include a wide range of dosage forms (creams, spray-type, foam, and gels) containing either lidocaine base or lidocaine hydrochloride, and to obtain a broad range in the delivery from the prototype formulations.

This study was conducted using procedures adapted from the FDA and AAPS Report of the Workshop on Principles and Practices of In Vitro Percutaneous Penetration Studies: Relevance to Bioavailability and Bioequivalence (Skelly et al., 1987). The clinically relevant dose of 5 mg/cm² was applied to dermatomed human abdominal tissue from a single donor obtained following elective surgery. The thickness of the tissue ranged from 0.025-0.038 inches (0.635-0.965 mm) with a mean+/−standard deviation in thickness of 0.031+/−0.004 inches (0.792+/−0.092 mm) and a coefficient of variation of 11.6%.

Percutaneous absorption was evaluated using this human abdominal tissue from a single donor, which was mounted in Bronaugh flow-through diffusion cells. The cells were maintained at a constant temperature of 32° C. by use of recirculating water baths. These cells have a nominal diffusion area of 0.64 cm². Fresh receptor phase, PBS with 0.1% sodium azide and 4% Bovine Serum Albumin, was continuously pumped under the tissue at a flow rate of nominally 1.0 ml/hr and collected in 6-hour intervals. The receptor phase samples were collected in pre-weighed scintillation vials; the post weights were taken following the study duration. Following the 24-hour duration exposure, the formulation residing on the tissue surface was removed by tape-stripping with CuDerm D-Squame stripping discs. The amount of Lidocaine residing in the epidermis, dermis, and receptor phase samples were properly labeled and were then sent to Pyxant Labs, Inc., an external contract bioanalytical laboratory, for subsequent analysis of Lidocaine content by LC/MS/MS and ultimate sample disposal.

Table 3 provides the composition of the formulations that were tested. The mass of Lidocaine per square centimeter of dosed tissue was calculated using the mass of Lidocaine in each sample divided by the area of skin exposed to the formulation.

Tissue permeation results were statistically evaluated using unpaired student's t-tests (significant differences between formulations were defined by a p-value of <0.05, at the 95% confidence interval).

Results

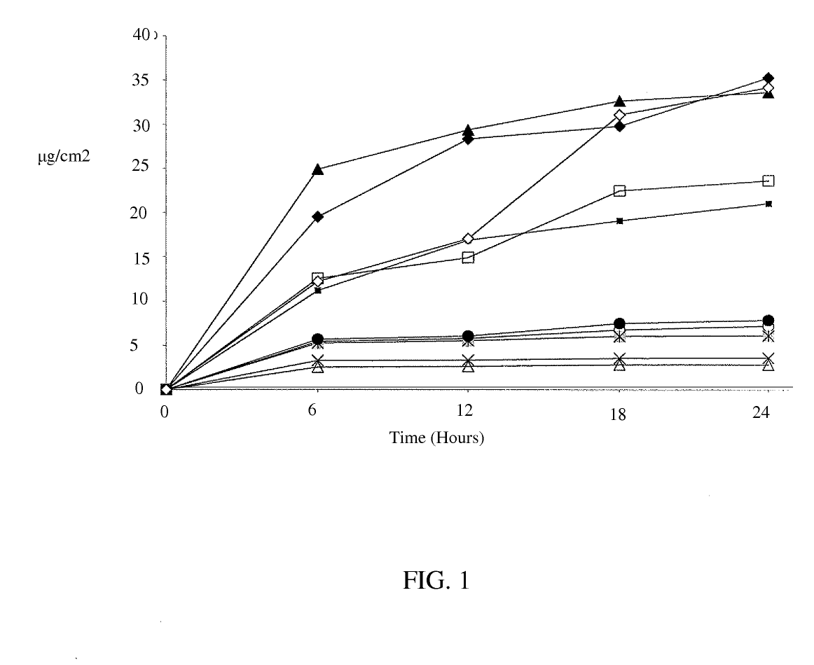

As shown in FIG. 1. Lidocaine delivery from all three gel (2749-30, 249-31, and 2749-32) candidates was equivalent or better than the compounding pharmacy formulation, and lidocaine delivery from two of the gels (2749-32 and 2749-30) was comparable to the LIDODERM® patch over a 24 hour period.

The LIDODERM®@patch demonstrated a more linear rate of drug permeation over the 24 hour period compared to the test formulations.

Formulations 2749-32 and 2749-30 delivered more lidocaine than all other formulations, including the LIDODERM® patch, from time of application to 6 hours. This indicates that these two formulations may have a faster onset of action and thus should provide more rapid pain relief.

Formulation 31 had comparable delivery profile to the compounding pharmacy cream. Prototype candidates containing Lidocaine HCl did not deliver as well as the formulations containing the free base.

Skin permeation (receptor phase levels) of Lidocaine ranged from 2.8 to 35 μg/cm² from Formulations 2749-72 and 2749-30, respectively. Formulations 2749-32 and 2749-30 had the highest permeation amount of Lidocaine with 34 and 35 g/cm², respectively. Lidocaine delivery from Formulations 2749-32 and 2749-30 were comparable to the LIDODERM® patch, 34 μg/cm². Tissue permeation of Lidocaine from the control formulations (Compounding Pharmacy Product and Lidocaine Patch) was 24 and 34 μg/cm² (equivalent to 1.4 and 0.68 percent of the applied dose of Lidocaine), respectively. Cutaneous delivery of Lidocaine following 24 hours exposure from Formulations 2749-32 and 2749-30 was comparable to the Lidocaine Patch. Skin permeation of Lidocaine from Formulations 2749-32 and 2749-30 as well as the LIDODERM® patch was significantly higher (p<0.05, unpaired student's t-test) than the Compounding Pharmacy Product, 24 μg/cm². Skin permeation from Formulation 2749-31 (21 g/cm²) was comparable to that of the Compounding Pharmacy Product.

The kinetic profile of tissue permeation is presented in FIG. 1 where the cumulative tissue permeation of Lidocaine in units of g/cm² is plotted against time in hours. Skin permeation of Lidocaine was almost complete following the first 12 hours of exposure following topical application of the semisolid dosage forms; whereas, the LIDODERM® patch demonstrated a more linear rate of drug permeation over the 24 hour duration of this study. Formulations 2749-32 and 2749-30 delivered more Lidocaine that all other formulations from time of application to 6 hours, the first time point of receptor phase collection, suggesting that these two formulations may have a faster onset of action, which should results in more rapid pain relief. Formulation 2749-31 had comparable Lidocaine delivery profile to the Compounding Pharmacy Product. The tissue penetration of Lidocaine was statistically evaluated using unpaired student's t-tests (significant differences between formulations were defined by a p-value of <0.05, at the 95% confidence interval).

Results

The results are shown in FIG. 1 and Table 4. FIG. 1 is a graph of the cumulative penetration of lidocaine through human skin, measured as micrograms lidocaine/cm², over time in hours. Table 4 shows the cumulative receptor phase levels of lidocaine in percent of applied dose.

Lidocaine-containing gels exhibited greater cumulative penetration of lidocaine than lidocaine HCl-containing creams and sprays. LIDODERM®, a commercially available patch comprised of an adhesive material containing 5% lidocaine, which is applied to a non-woven polyester felt backing and covered with a polyethylene terephthalate (PET) film release liner, is applied only once for up to 12 hours in a given 24 hour period.

The Lidocaine Gel formulations 2749-32 and 2749-30 gave the highest levels of Lidocaine delivery of all semisolid dosage forms tested and the total amount of Lidocaine delivered over 24 hours from these two gel formulations was comparable to that achieved with the marketed LIDODERM® patch. Formulations 2749-32 and 2749-30 delivered more lidocaine than all other formulations, including the LIDODERM® patch, from time of application to 6 hours. This indicates that these two formulations may have a faster onset of action and thus should provide more rapid pain relief.

Comparable Lidocaine delivery and kinetic profile to the Compounding Pharmacy Product were achieved with Formulation 2749-31.

TABLE 3
Formulation Compositions

| | | | | Lidocaine | Spray- | | | |
|---|---|---|---|---|---|---|---|---|
| Ingredients | Lidocaine Gel | | | HCI Gel | type | Foam | Creams | |
| (% w/w) | 2749-30 | 2749-31 | 2749-32 | 2749-28 | 2749-72 | 2749-52 | 2749-37 | 2749-38 |
| Lidocaine | 40 | 40 | 40 | | | | | |
| Lidocaine HCl | | | | 40 | 40 | 40 | 40 | 40 |
| Isopropyl Myristate | | | 10 | | | | | |
| Transcutol P | 10 | 20 | 10 | | | | | |
| Propylene glycol | 10 | 10 | 20 | | | | | |
| Dimethyl isosorbide | 10 | 10 | | | | | | |
| Ethyl acetate | 10 | | | | | | | |
| Isopropyl alcohol | 18.8 | | | 10 | | | | |
| Ethanol 200 proof | | 18.8 | 18.8 | | | | | |
| Hydroxypropyl cellulose, HXF Pharm | 1.2 | 1.2 | 1.2 | | | | | |
| Hydroxyethylcellulose, 250HHX Pharm | | | | 1 | 1 | | 0.5 | 0.5 |
| Benzyl Alcohol | | | | 2 | 2 | 2 | | |
| Propylene Glycol | | | | 10 | 10 | 5 | 5 | 5 |
| Glycerin | | | | | | 3 | | 2 |
| Sorbitol 70% | | | | 3 | | | | |
| Polysorbate 80 | | | | | 1 | | | |
| Sodium Laureth Sulfate | | | | | | 5 | | |
| Disodium Laureth Sulfosuccinate | | | | | | 5 | | |
| Diisopropyl Adipate | | | | | | | | 10 |
| White Petrolatum | | | | | | | 3 | |
| Light Mineral Oil | | | | | | | 10 | |
| Pemulen TR1 | | | | | | | 0.3 | 0.3 |
| Sorbitan Monoleate | | | | | | | 0.4 | 0.4 |
| Methylparaben | | | | | | | 0.17 | 0.17 |
| Propylparaben | | | | | | | 0.03 | 0.03 |
| Purified Water | | | | qsad | qsad | qsad | qsad | qsad |

TABLE 4
Cumulative Receptor Phase Levels of Lidocaine in Percent of
Applied Dose

| | | | Hour(s) | | | | |
|---|---|---|---|---|---|---|---|
| Formulation ID | | | 0 | 6 | 12 | 18 | 24 |
| A) | ID: 2749-30 | Mean | 0 | 1.09 | 1.59 | 1.67 | 1.98 |
| | | SD | 0 | 0.25 | 0.17 | 0.18 | 0.46 |
| | | % CV | 0 | 22.95 | 10.94 | 10.88 | 23.46 |
| B) | ID: 2749-31 | Mean | 0 | 0.63 | 0.94 | 1.07 | 1.18 |
| | | SD | 0 | 0.30 | 0.49 | 0.47 | 0.46 |
| | | % CV | 0 | 47.53 | 51.74 | 44.26 | 39.04 |
| C) | ID: 2749-32 | Mean | 0 | 1.47 | 1.73 | 1.92 | 1.98 |
| | | SD | 0 | 0.37 | 0.38 | 0.42 | 0.44 |
| | | % CV | 0 | 25.08 | 21.96 | 22.03 | 22.36 |
| D) | ID: 2749-28 | Mean | 0 | 0.16 | 0.17 | 0.18 | 0.18 |
| | | SD | 0 | 0.13 | 0.13 | 0.13 | 0.13 |
| | | % CV | 0 | 78.68 | 77.71 | 73.03 | 72.28 |
| E) | ID: 2749-72 | Mean | 0 | 0.13 | 0.14 | 0.15 | 0.15 |
| | | SD | 0 | 0.05 | 0.05 | 0.05 | 0.05 |
| | | % CV | 0 | 35.20 | 36.06 | 35.99 | 35.30 |
| F) | ID: 2749-52 | Mean | 0 | 0.28 | 0.30 | 0.36 | 0.38 |
| | | SD | 0 | 0.15 | 0.16 | 0.20 | 0.21 |
| | | % CV | 0 | 53.25 | 53.58 | 54.70 | 54.20 |
| G) | ID: 2749-37 | Mean | 0 | 0.29 | 0.31 | 0.33 | 0.34 |
| | | SD | 0 | 0.23 | 0.24 | 0.26 | 0.26 |
| | | % CV | 0 | 79.61 | 77.82 | 77.47 | 76.29 |
| H) | ID: 2749-38 | Mean | 0 | 0.28 | 0.30 | 0.37 | 0.39 |
| | | SD | 0 | 0.19 | 0.19 | 0.22 | 0.22 |
| | | % CV | 0 | 67.57 | 63.34 | 59.77 | 57.39 |
| I) | ID: Compounding | Mean | 0 | 0.73 | 0.87 | 1.33 | 1.39 |
| | Pharmacy Product | SD | 0 | 0.14 | 0.15 | 0.17 | 0.14 |
| | | % CV | 0 | 18.92 | 17.46 | 12.76 | 10.40 |
| J) | ID: Lidocaine Patch | Mean | 0 | 0.24 | 0.34 | 0.62 | 0.68 |
| | | SD | 0 | 0.04 | 0.03 | 0.18 | 0.21 |
| | | % CV | 0 | 14.36 | 7.54 | 29.75 | 30.68 |

1-11. (canceled)

12. A topical formulation for treating pain comprising a local anesthetic free base caine alkaloid and a pharmaceutically acceptable topical carrier in a lotion, cream, dispersion, or gel, wherein the local anesthetic free base caine alkaloid is present in an amount from greater than 20% by weight to about 60% by weight of the formulation.

13. The topical formulation of claim 1, wherein the local anesthetic free base caine alkaloid is selected from the group consisting of dibucaine, bupivacaine, etidocaine, tetracaine, lidocaine, and xylocaine.

14. The topical formulation of claim 1, wherein the local anesthetic free base caine alkaloid is lidocaine.

15. The topical formulation of claim 1, wherein the formulation is a gel.

16. The topical formulation of claim 1, wherein the local anesthetic is present in an amount from greater than 20% by weight to 40% by weight of the formulation.

17. The topical formulation of claim 1, wherein the local anesthetic is present in an amount of approximately 40% by weight of the formulation.

18. A method of treating pain, comprising administering to a site of or adjacent to the pain in a patient an effective amount of the topical formulation of claim 1 to thereby treat the pain.

19. The method of claim 18 wherein the pain is neuropathic pain.

20. A topical formulation for treating pain, comprising:
(a) from about 40% by weight to about 60% by weight of a local anesthetic that is lidocaine in free base form; and
(b) a pharmaceutically acceptable topical carrier comprising a glycol and isopropyl myristate;
the topical formulation being a gel, lotion, or cream.

21. The topical formulation of claim 20, wherein the glycol is propylene glycol.

22. The topical formulation of claim 21, further comprising diglycol monoethyl ether.

23. The topical formulation of claim 22, further comprising a modified cellulose.

24. The topical formulation of claim 23, wherein the modified cellulose is hydroxypropyl cellulose.

25. The topical formulation of claim 23, further comprising an alcohol.

26. The topical formulation of claim 24, further comprising ethanol.

27. The topical formulation of claim 20, wherein the formulation comprises about 40% by weight lidocaine in free base form.

28. The topical formulation of claim 22, wherein the formulation comprises about 40% by weight lidocaine in free base form.

29. The topical formulation of claim 26, wherein the formulation comprises about 40% by weight lidocaine in free base form.

30. The topical formulation of claim 20, wherein the formulation is a gel.

31. The topical formulation of claim 22, wherein the formulation is a gel.

32. The topical formulation of claim 26, wherein the formulation is a gel.

33. The topical formulation of claim 29, wherein the formulation is a gel.

34. The topical formulation of claim 6, wherein the formulation is anhydrous.

35. A method of treating pain, comprising administering to a site of or adjacent to the pain in a patient an effective amount of the topical formulation of claim 20 to thereby treat the pain.

36. The method of claim 35, wherein the pain is neuropathic pain.

37. A method of treating pain, comprising administering to a site of or adjacent to the pain in a patient an effective amount of the topical formulation of claim 22 to thereby treat the pain.

38. The method of claim 37, wherein the pain is neuropathic pain.

39. A method of treating pain, comprising administering to a site of or adjacent to the pain in a patient an effective amount of the topical formulation of claim 18 to thereby treat the pain.

40. The method of claim 39, wherein the pain is neuropathic pain.

41. The method of claim 39, wherein the pain is due to a herpes zoster infection.
